(19) United States
(12) Patent Application Publication
(10) Pub. No.: US 2013/0159712 A1
(43) Pub. Date: Jun. 20, 2013

(54) SYSTEM AND METHOD FOR VERIFYING AND MANAGING DISTRIBUTION OF PRODUCTS

(71) Applicant: Pharmasecure, Inc., Lebanon, NH (US)

(72) Inventors: Nathan J. Sigworth, Hamden, CT (US); N. Taylor Thompson, Greensboro, NC (US)

(73) Assignee: PHARMASECURE, INC., Lebanon, NH (US)

(21) Appl. No.: 13/706,014

(22) Filed: Dec. 5, 2012

Related U.S. Application Data
(63) Continuation-in-part of application No. 12/973,879, filed on Dec. 20, 2010.
Application No. 12/973,879 is a continuation-in-part of application No. 12/973,879, filed on Dec. 20, 2010.
(60) Provisional application No. 61/567,049, filed on Dec. 5, 2011.
(60) Provisional application No. 61/313,265, filed on Mar. 12, 2010.

(51) Int. Cl. H04L 9/32

(52) U.S. Cl. CPC H04L 9/32
USPC 713/168

(57) ABSTRACT

A system and method for verifying, validating and otherwise managing distribution of products and medicines reduces the instances of counterfeit medicines. A pharmaceutical company typically provides medicines/products to users either directly or through representatives for the pharmaceutical company. The products have associated identifying or authentication codes that are used to authenticate the validity of the medicine/product. The system encrypts and decrypts code data employing appropriate client- and server-based applications to securely manage and print the authenticating code data. A covert identification technique, such as a special ink or material can provide an additional level of security in authenticating the medicine to ensure it is not counterfeit. The special ink or material can be tested locally by the user or sent to a remote location for testing to ensure accuracy of the medicine/product.

### RELATED APPLICATIONS

This application claims the benefit of U.S. Provisional Application Ser. No. 61/567,049, filed Dec. 5, 2011, entitled SYSTEM AND METHOD FOR VERIFYING AND MANAGING DISTRIBUTION OF PRODUCTS, the entire disclosure of which is herein incorporated by reference. This application is also a continuation-in-part of U.S. patent application Ser. No. 12/973,879, filed Dec. 20, 2010, entitled SYSTEM, METHOD AND INTERFACE DISPLAY FOR VERIFYING AND MANAGING DISTRIBUTION AND SALES OF MEDICINE, the entire disclosure of which is herein incorporated by reference, which claims the benefit of U.S. Provisional Patent Application Ser. No. 61/313,265, filed Mar. 12, 2010, entitled SYSTEM, METHOD AND INTERFACE DISPLAY FOR VERIFYING AND MANAGING DISTRIBUTION AND SALES OF MEDICINE, the entire disclosure of which is herein incorporated by reference.

### FIELD OF THE INVENTION

This invention relates to systems and methods for managing medicines and other pharmaceutical products provided by a pharmaceutical company. More particularly, to systems and methods for validating and/or verifying the authentication of medicines and other pharmaceutical products.

### BACKGROUND OF THE INVENTION

There is a major problem related to the distribution, sales and use of counterfeit drugs and other pharmaceutical products. In many countries around the world, medicines that are distributed and sold are fake, i.e. counterfeit. These counterfeit medicines are deliberately and fraudulently mislabeled with respect to identity and/or source. They can range from unknown random combinations of toxic substances to ineffective, inactive preparations, such as lacking an active ingredient. The results of counterfeit drugs are that either there is an ineffective treatment, a harmful outcome, or even worse, death, for a patient or end user of the medicine.

In January 2010, the World Health Organization published an article on counterfeit drugs, describing the major health concern related with these illegal medicines. One counterfeit medicine for lowering blood sugar levels resulted in two deaths and nine hospitalizations in 2009 alone. In the United Republic of Tanzania in 2009, an antimalarial drug (Metakelfin), however lacking sufficient level of the active ingredient, was discovered in 40 pharmacies. This is particularly a problem in countries having weaknesses in their regulatory and enforcement systems. An estimated 1 in 4 packets of medicine sold in the street markets in developing countries is believed to be counterfeit. Over 50% of the medicines purchased over the Internet are from illegal sites that conceal their physical address, have been found to be counterfeit.

More generally, in this environment (and even that of more-developed countries), a pharmaceutical manufacturer typically has little to no interaction or review of the sales of their medicines. After distributing their products to representatives or directly to end users, the pharmaceutical company does not communicate with the user thereafter. Likewise, the medical distributor simply sells products, with no feedback from users or sales information pertaining to the users. There is no interaction between the users of the medicines and the providers (manufacturers or distributors) or the products. There is not an effective way of eliminating these counterfeit medicines, accordingly there is a need for a system that verifies the validity of medicines, for the sake of all parties involved, particularly the end user of the medicine.

Accordingly, there is a need for a system and method for accurately verifying the validity of medicines to ensure they are not counterfeit. The system and method should allow pharmaceutical companies and providers to observe when and where their products are selling. A desirable system would help reduce the availability of counterfeit medicines by allowing users to ensure a product is valid prior to use and/or consumption. There is a need for a system that obtains information pertaining to the users of the products to further analyze sales and distribution based on that information.

Moreover, distribution data in many emerging markets is often not accurate and/or is delayed. In general, drug distribution information is collected from pharmacy inventories and doctor prescriptions. In markets where pharmacies do not keep detailed real-time electronic inventories or where patients regularly purchase drugs without a prescription, accurate distribution data is difficult, if not impossible, to gather. Any data that is gathered disadvantageously has a delay between the sale of medicines and the time the data is available to manufacturers and other parties involved in drug delivery.

Furthermore, longitudinal research among consumers is impossible or difficult to carry out. There is a need for a method for enabling a consumer to be contacted as a follow-up after they purchase a medicine.

### SUMMARY OF THE INVENTION

A system and method verifies the validity of medicines, as well as the tracking, monitoring, and otherwise managing medicines. The system and method performs related analysis of the data of product sales and distribution data. The system allows pharmaceutical companies to manage the sales and distribution of products based on a code associated with a particular package of medicine or the product itself while allowing users to verify the validity of the medicine or product. Each medicine includes an identifying product code used in managing the distribution and sales of the products, thereby identifying a particular product of a batch of products. Accordingly, each individual package or container of medicine has its own unique authentication code to identify the particular medicine. The individual package or container of medicine can also be in the form of a box, a blisterpack, a vial, a bottle, and a dose of medicine. These codes can be uploaded by users and pharmaceutical company representatives through any number of communication devices, including via a text message, a Smartphone application, the Internet in general, or any other appropriate network. The code can be provided directly on an exterior surface of a bottle or other container for the medicine so it is readily available to a user.

The system includes a verification server that manages and controls the code data and information regarding sales of medicines. The verification server further analyzes the sales of medicines, verifying the validity of the medicines. The system further includes a management server that includes a plurality of applications running thereon for further managing the sales of medicines and related statistical data. The system allows pharmaceutical companies to send response and verification messages to users and representatives of the products. This improves feedback to users and pharmaceutical communication overall. The verification messages can be the same form as the codes that are uploaded by the users, or can be in a different form. For example, the verification messages can be in the form of text messages, electronic mail communications, voice mail messages, messages/interaction with a Smartphone application, the Internet in general, or any other appropriate message provided through a network.

An overall system printing server which can include the verification server and management server, or be a distinct server) allows for the management and creation of code data that can be provided on a pharmaceutical product instead of, or in addition to, traditional identifying codes on the product. The system includes a database having a plurality of authentication codes stored thereon and employs an encryption process to encrypt at least some of the authentication codes into a secure format for upload to a secured network. Client applications are authenticated to the secured network to receive the securely formatted data file. The securely formatted data file is decrypted by the client application and sent to printers such that the authentication code data is printed on the medicine/product and the pharmaceutical company does not have an unsecure record of the printing. This enhances the security of the code data and overall validation of products and/or medicines.

A method for collecting and disseminating real-time market data of product distribution commences with printing an authentication code on a package. The authentication code corresponds to production data, such as the time and place of production, drug information and product batch information. The production data is stored in an authentication database. Thereafter, a user-provided authentication message is received that contains authentication data. The authentication data is registered and compared with the production data to determine and/or verify the distribution data. The distribution data is then combined with other predetermined data (e.g. disease incidents) and running regressions to derive factors that create demand for a product. These factors, as well as distribution data, can then be presented to the manufacturers. Advantageously, this data can be presented anonymously to the manufacturers, to maintain the privacy of users.

A method for conducting longitudinal consumer research and communication commences by printing an authentication code on a package. An authentication message is received from the user, containing authentication data for a user to verify a particular product or medicine. The user information and authentication message is stored in a user database. Once the medicine or product has been authenticated, the user is contacted through a message. The message can be used to either remind the user to take his or her medicine or to ascertain the status of use of the medicine by the user.

In an exemplary embodiment, a blood pressure patient purchases a medicine and authenticates it through authentication messages. Because the exact package, batch, drug, and date of authentication is known, a call or SMS message can be sent to the consumer a specified amount of time after the first authentication. This message can be used to determine if the consumer is still taking the medicine, if they have stopped taking it, or if they have switched to a different brand. This information is extremely valuable to manufacturers and brand owners and this is a novel way of interacting with consumers.

A method for providing real-time interaction with consumers comprises providing a unique authentication code on an individual product. Authentication message are received from users and stored in a user database. A menu of application is provided to the user to facilitate the communication between manufacturers and consumers. Information from the applications is then compiled in aggregate to provide aggregated information to a user.

### BRIEF DESCRIPTION OF THE DRAWINGS

The invention description below refers to the accompanying drawings, of which:

FIG. 1 is an overview block diagram of a system for managing the sales and distribution of medicines, according to an illustrative embodiment;

FIG. 2 is a flow diagram showing an overall procedure for managing the sales and distribution of medicines, according to the illustrative embodiments;

FIG. 3 is a flow diagram detailing a procedure for the implementation of the dashboard user interface, according to the illustrative embodiments;

FIG. 4 is a flow diagram showing a procedure for the implementation of a response controller and manager for the system for managing medicines, according to the illustrative embodiments;

FIG. 5 is an overview block diagram of a system for serialization and verification of pharmaceutical products, in accordance with an illustrative embodiment;

FIG. 6 is an overview block diagram of a system for printing code data and showing the flow of data pertaining thereto, according to an illustrative embodiment for code generation;

FIG. 7 is an architectural diagram of the various operations performed by the client and server, according to the illustrative embodiments;

FIG. 8 is a flow diagram showing a procedure for collecting and disseminating real-time market data of product distribution, according to the illustrative embodiment;

FIG. 9 is a flow diagram showing a procedure for conducting consumer research and communication, according to the illustrative embodiments;

FIG. 10 is a flow diagram showing a procedure for providing real-time interaction with consumers, according to the illustrative embodiments;

FIG. 11 is a flow diagram showing a procedure for performing dual-authentication using an authentication code and a covert authentication element, according to the illustrative embodiments;

FIG. 12 is a flow diagram showing a procedure for providing real-time information to consumers, according to the illustrative embodiments;

FIG. 13 is an exemplary screen display of an authentication screen for authenticating a particular product, according to an illustrative embodiment;

FIG. 14 is an exemplary screen display of the authentication screen showing a response message based upon an incorrect authentication code, according to the illustrative embodiment;

FIG. 15 is an exemplary screen display of the authentication screen showing a response message based upon an oververified code, according to the illustrative embodiment; and

FIG. 16 is an exemplary screen display of the authentication screen showing a response message based upon an incorrect confirmation code, according to the illustrative embodiment

### DETAILED DESCRIPTION

A system and method for verifying the validity of medicines and/or pharmaceutical products tracks, monitors and otherwise manages the distribution of medicines and/or pharmaceutical products. Pharmaceutical companies are able to monitor the distribution of their medicines and products and users are also able to verify the validity of the medicine/product. The terms “medicine” and “product” as used herein are intended to cover either a medicine or a product, or both, or any other package of a pharmaceutical product that is “taken”, consumed or otherwise used by a user and known in the art. The validity is determined through use of an identifying code provided by the pharmaceutical company or an authentication code printed directly on the medicine/product securely managed by a party other than the pharmaceutical product to maintain the security of the authentication codes and reduce risk of outsider obtaining the code data.

Monitoring Distribution of Products Using Unique Product Codes

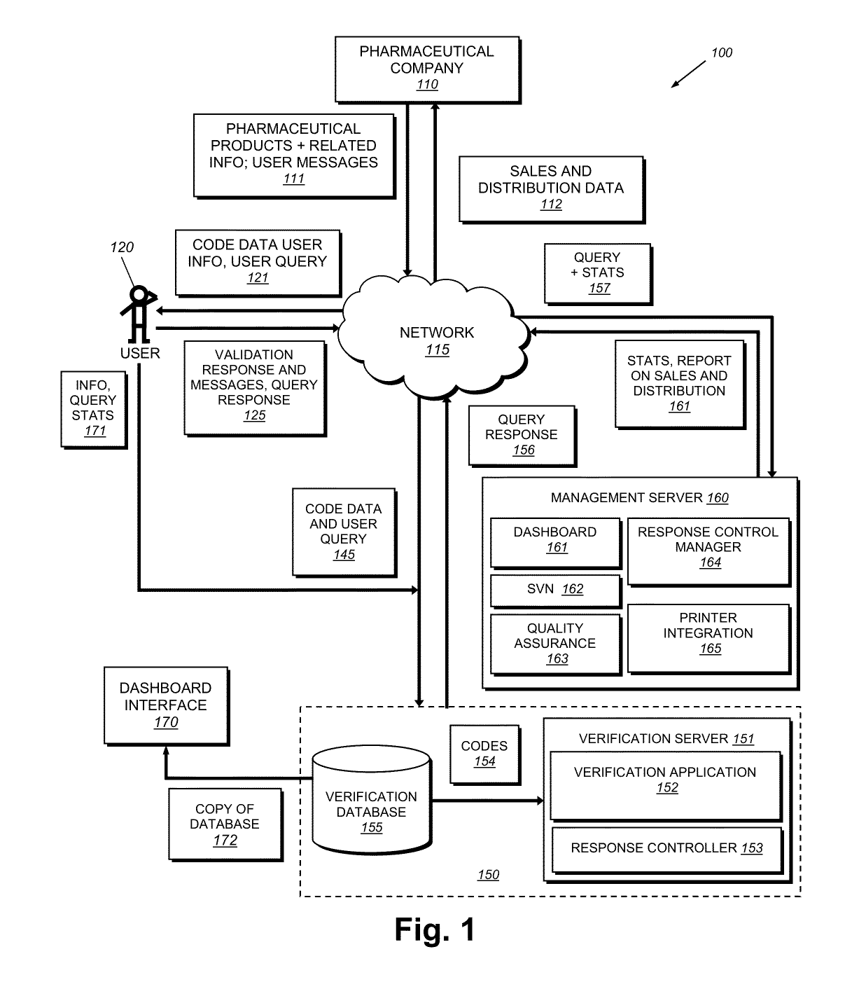

There is provided a system and method for verifying the validity of medicines, as well as tracking, monitoring, and otherwise managing medicines, and related analysis of the sales and distribution data. As shown in FIG. 1, a medicine management system 100 is provided in which a pharmaceutical company 110 provides medicines to users and consumers. “Users” as defined herein represent either end users of the medicines, or representatives for the pharmaceutical company that assist in the distribution and sales of the medicines to end users.

As shown in FIG. 1, a pharmaceutical company 110 transmits the products and related information (including codes identifying the products) via datastream 111 to a network 115. In an illustrative embodiment, the verification codes which identify a particular product are provided by the pharmaceutical company. Each individual medicine or product (e.g., bottle, vial or container) includes an identifying verification product code used in managing distribution and sales, and is also used to identify a particular product. In alternate or additional embodiments, as described in greater detail hereinbelow, an authentication code can also be printed directly onto the product or medicine to assist in verifying the particular product or medicine. The network 115 can comprise any appropriate communication network including a local area network (LAN), the broad worldwide Internet, cellular telephones used through a cellular tower network or any other appropriate network or secured connection. As described in greater detail hereinbelow, the system provides the pharmaceutical company with feedback based on the distribution and, more importantly, sales of the medicines via datastream 112. This information allows the pharmaceutical company to develop user messages based on this information, which is transmitted to the network via datastream 111, to be sent to a particular user or consumer. The code data uploaded to the system, as well as the response “verification” messages transmitted to the user, can be in any appropriate form of communication through any network. For example, the messages can be standard text message (SMS (short messaging service), MMS (multimedia messaging service) or other, Smartphone application messages, USSD (Unstructured supplementary service data) having a particular series of characters and letters used to authenticate a medicine (such as #AUTHENTICATE*CODE DATA), using a Twitter handle to tweet the authentication code to the appropriate entity, or through the Internet in general via an electronic internet-based message.

A user 120 of the system 100 transmits code data about the individual medicine, as well as user information, containing particular information about the user via datastream 121. The user can be an end user or consumer, seeking to verify the validity and safety of their medications. A user transmits the code data to verify the validity of the individual medicine the user is seeking to authenticate, based upon feedback from the various components of the system 100.

According to the system 100, the user submits a query via datastream 121 to the network 115 to perform a call in function to verify the validity, or perform one of a plurality of other applications pertaining to the system. The code data and user query are transmitted via datastream 145 to a verification system 150. The verification system includes a verification server 151 that has a verification application 152 and response controller 153 running thereon. The verification server 151 receives the query responses and codes to perform appropriate control and verification of data to verify or authenticate the validity of the medicines. The verification server 151 also stores the codes 154 into the secure verification database 155, within the verification system 150. The statistics and reports on sales and distribution of the medicines generated by the verification system are transmitted back to the network 115 via datastream 156. These are then transmitted back to a user 120 via datastream 125 and a pharmaceutical company 110 via datastream 112. While the various servers and/or applications are depicted at specific locations and perform specific tasks, the relative positions and division of tasks between the various entities is highly variable.

User queries and statistics are transmitted via datastream 157 to a management server 160. The management server 160 includes a plurality of applications that perform a multitude of functions. The management server 160 performs one of a multitude of functions, and this information, as statistics, reports on sales and distribution, or other pertinent data, is transmitted via datastream 161 to the network 115, to be further transmitted to pharmaceutical companies 110 and users 120.

According to an illustrative embodiment, on a periodic basis, the secure verification database 155 selectively copies itself (no codes are transferred, only the verification data) to provide a copy to the users, for example, as a dashboard database at a dashboard interface. The decoupling between codes and verification data secures the system by transferring only verification data, and not codes, to the dashboard interface. A dashboard database (the copy of the database without codes) is transmitted to the dashboard user at a dashboard interface 170 via datastream 172. These updated statistics therefore can reside on the management server 160 through the dashboard user interface or other appropriate server, application, database or computer-readable non-transitory storage medium. A query made by the user, via datastream 171, at the dashboard user interface 170 reflects the updated statistics data.

The verification server and the management server can comprise any appropriate server or computing architecture, including a computer program running as an application or other service, a physical computer dedicated to running applications, and/or a software/hardware system of components, as described herein and otherwise readily apparent to those of ordinary skill.

Referring further to FIG. 1, the management server 160 includes a plurality of applications, functions and/or additional servers for performing the various tasks of the system for managing sales of medicines. These applications include, for example, a dashboard application 161, SVN (Version Control) application 162, Quality Assurance application 163, Response Control Manager application 164, and Printer Integration application 165. It is expressly contemplated that while these are shown and described as applications residing on the management server, each can be represented as a separate server or as a virtual machine, or an application running on a server or virtual machine. Likewise, the distribution of tasks between the various applications and/or servers is highly variable. Furthermore, the placement and location of servers and applications is highly variable.

The dashboard 161 is an analytics tool used to provide the total verifications, daily verifications, regional verifications, product verifications and cross-sell verifications, among others, for the system for verifying medicines. The SVN application 162 is a version-control system that is used to maintain current and historical versions of files. Can be used for quality assurance and other production environments. The quality assurance application 163 analyzes data to ensure quality assurance of the overall system. The response control manager 164 coordinates with the verification server 150 to manage responses based on products and/or batches of products. The printer integrator 165 can be a server or application that connects with the pharmaceutical company to transfer codes online. The printer integrator 165 also coordinates with the verification system 150 to verify the codes.

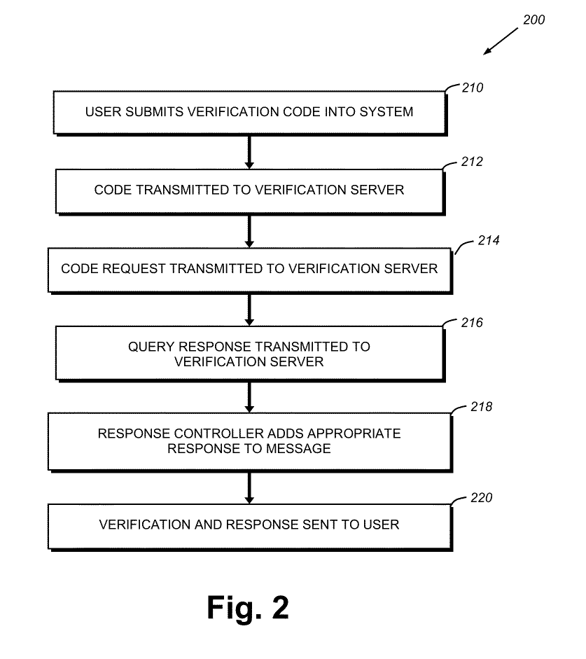

The statistics and reports on the sales and distribution of the medicines are generated by the verification system according to the procedures and methods disclosed herein and discussed in greater detail with reference to the flow charts of FIGS. 2-4.

Referring now to FIG. 2, a procedure 200 is shown for the user interaction with the system for verification and managing medicine sales. The user verification procedure 200 begins at step 210 when the user submits a verification code into the system. This verification code is representative of an individual medicine or can be used to designate a batch (group) of medicines. This code is then transmitted to the verification server, as well as information about the user, at step 212, to verify the validity of the medicines. The procedure then transmits a code request to the verification database at step 214 to obtain information about the medicine. This information is used in tracking the sales of the medications. The query response is then transmitted back to the verification server at step 216.

Based upon the query response, the verification server prepares a message for the user and the response controller application (running on verification server) adds an appropriate response to the message at step 218. The verification and response are then sent to the user via the network at step 220. These are used to communication a verification (or lack thereof) to a user. The verification messages can be presented to the user through any appropriate system described herein, including text messages through a cellular telephone network, Smartphone application messages, and internet-based messages, among others.

There can be at least three types of responses generated for a user. The first is a “valid” response for up to “n” notifications, where “n” is any number of medicines. An example of this first response is: “MCN labs authenticates genuine product of [Product Name], production data [Product Number]. Thank you for your patronage. Service provided by PharmaSecure.” The second is an “invalid” response, which is returned if the customer enters an invalid code. An example of this second response is: “The code typed ‘----’ is incorrect. Please retype the code or contact chemist for further assistance. Service provided by PharmaSecure.” The third is an “oververified” code, in which the number of verifications is excessive for that particular code, thereby indicating it is not a valid medicine. An example of this third response: “Warning! Expired code! Please contact the chemist for further assistance. Service provided by Pharmasecure.” Other messages and variations of those above can be provided as appropriate and within ordinary skill to the users as desired by a particular pharmaceutical company or other interested party.

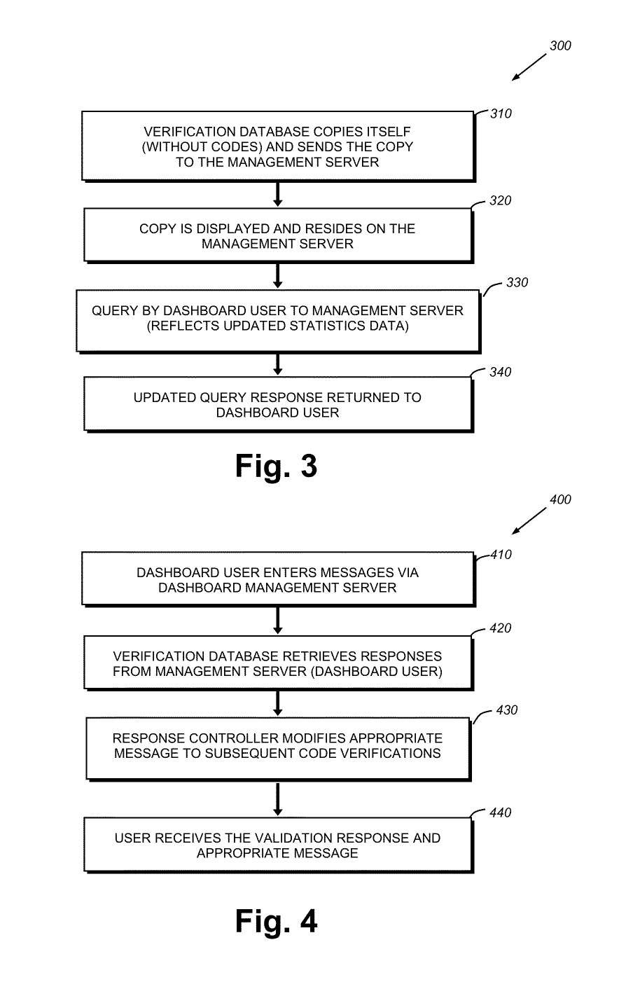

FIG. 3 shows a dashboard user procedure 300 for interaction with the management server according to an illustrative embodiment. In an illustrative embodiment, the verification database copies itself and sends a copy to the management server at step 310. The copy is displayed and resides on the management server at step 320. This allows the updated statistics to reside on the management server. A query for updated data is sent to the management server at step 330 to reflect the updated statistics data on the dashboard user. The dashboard user requests updated data from the management server at step 330. The updated query response is then returned to the dashboard user at step 340. Therefore, updated data is present to the dashboard user.

FIG. 4 details a message management procedure 400 according to the illustrative system for managing medicine sales. The procedure 400 begins at step 410 when a dashboard user enters messages via the dashboard application on the management server, for example a request for verification of the validity and safety of a medicine. The messages include verification, error or expired response messages. The verification database then retrieves responses from the management server at step 420. These are pulled from the management server by the verification database on a periodic basis.

Then at procedure step 430 the response controller of the management server modifies an appropriate message for the dashboard user to include subsequent code verifications. The management server includes applications for performing various statistical analysis and review of the sales of the medicines. These are included in the response to the user to provide a message with details on the particular statistical data of the sales of the medicines. The user then receives the validation response and appropriate message at step 440, thereby validating (or finding a lack of validation) for the medicine.

Serialization, Verification and Printing Authentication Codes

To further improve security in managing the validity of medications and other pharmaceutical products, a product can be serialized, such as by providing an authentication code on the product. This authentication code can be in addition to, or in lieu of, the traditional codes and identifying information on a pharmaceutical product. As used herein, “authentication code” refers to a unique code generated specifically for an individual medicine or product for authenticating the validity of the particular medicine or product. The authentication code can also refer to a code applied to a package containing a group of individually packaged and coded medicines. The authentication code is used to identify a particular individual medicine, or group (e.g., package) of medicines, as desired, and can be implemented to verify distribution of medicines and products at various stages during the distribution process. The term “serialization” and variations thereof, as used herein, refer to the means by which an authentication code is assigned to (or otherwise provided or printed on) a particular pharmaceutical product or other object (e.g., a label).

According to an illustrative embodiment, the authentication codes can further be encrypted, or otherwise manipulated, encoded, enciphered, or generated, such that even the provider of the product is not aware of the exact code that has been provided (e.g., printed) on the product, thereby further enhancing the security and protection of code data and to decrease the likelihood and ability of malicious parties to reproduce the code data. This furthermore allows data to be gathered and distributed anonymously to protect the privacy of users. Moreover, as described in greater detail hereinbelow, a covert or forensic type of technology can also be provided on the pharmaceutical product to ensure that it is the true and actual pharmaceutical product and not a counterfeit product. For example, the code can be printed in a special ink or on a particular material that can be verified independently of the authentication code to provide additional, secondary, authentication of the medicine or product.

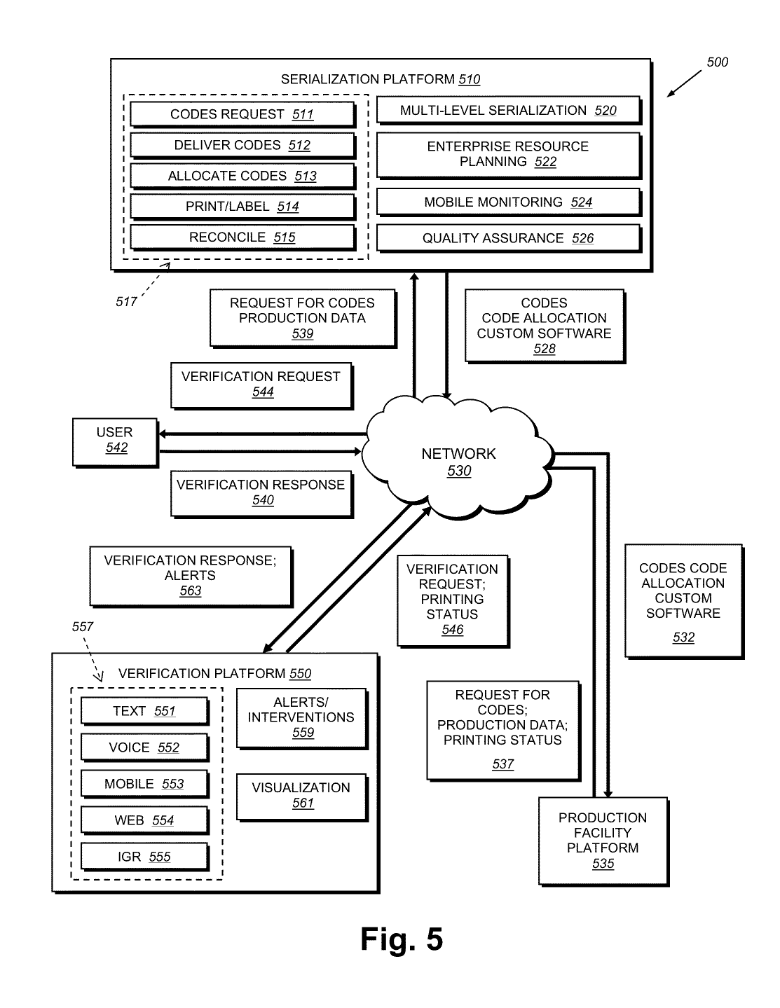

Reference is now made to FIG. 5, showing an overview block diagram of a system for serialization and verification of pharmaceutical products and other objects, in accordance with an illustrative embodiment. As shown, the serialization and verification system 500 includes a serialization platform 510 and a verification platform 550. The term “platform” is used herein to cover the application(s), server(s), tools and other features or functions available within a group or groups to carry out the functions herein for serialization and verification of pharmaceutical products and other objects. The platform can comprise any appropriate server or computing architecture, including a computer program running as an application or other service, a physical computer dedicated to running applications, and/or a software/hardware system of components, and any other variation or combination thereof, as described herein and otherwise readily apparent to those of ordinary skill.

The serialization platform 510 generates, affixes or otherwise provides authentication codes for pharmaceutical products and other objects. As shown, the serialization platform 510 includes a plurality of applications, functions and/or additional servers for performing various tasks or modules (511, 512, 513, 514, 515, 520, 522, 524, 526) during serialization. The term “module” refers to an application, tool, function, and/or additional server that performs various tasks during serialization and/or verification of the authentication codes, and is used interchangeably herein with “application” and “tool”. The serialization platform 510 includes applications or tools for performing the serialization tasks of: Codes Request 511, Deliver Codes 512, Allocate 513, Print/Label 514 and Reconcile 515. The group of serialization tasks 517, comprising the Codes Request 511, Deliver Codes 512, Allocate 513, Print/Label 514 and Reconcile 515, provides a complete serialization solution. A production facility (or printing facility/application) is able to customize the tasks available to them on their serialization platform. For example, in an illustrative embodiment, a production facility or printing platform can implement the tasks of Codes Request 511, in which the production facility securely places a request for a lot of unique codes and can track request status in real-time, and Deliver Codes 512, in which the serialization platform 510 delivers the codes securely to the plant (or other production facility) in standard formats for printing. In accordance with an illustrative embodiment, the production facility or printing platform can also implement Allocate 513, which allocates unique codes to packaging, and is capable of supporting multiple-location production management. The implemented feature of Allocate 513 provides line management, access control, audit trails and real-time status reporting without requiring a server in each individual location.

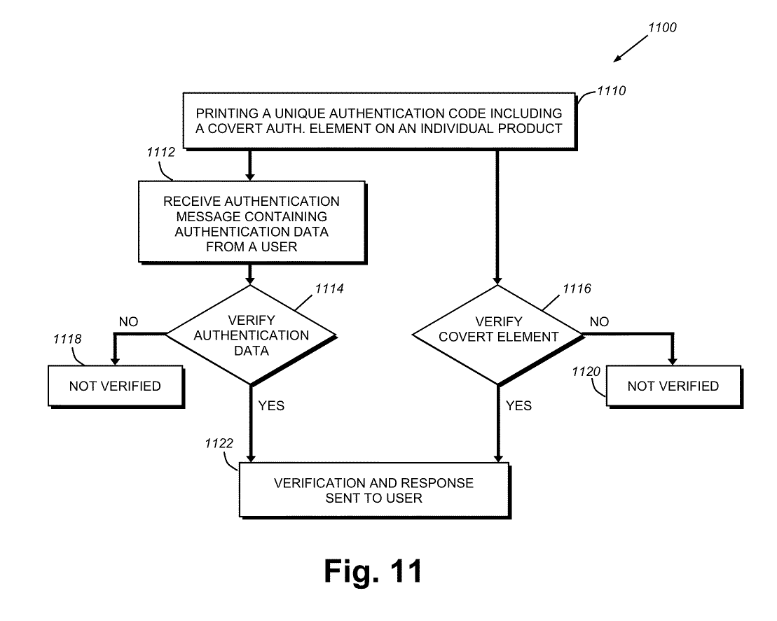

In accordance with an illustrative embodiment, the Print/Label application 514 can be implemented so the codes can be delivered to a location in an encrypted, enciphered or otherwise manipulated format, and appropriate customized software can be provided to facilitate printing of codes that are in unconventional formats by interfacing with the serialization platform and the production facility platform (or other location). The Print/Label application 514 securely prints codes on different levels of packaging at production speeds and uses sophisticated middleware to interface between the serialization platform and the printing application(s). The middleware allows for line integration with a variety of printers, and is software that is customized for specific printer models and protocols which are deployed on production lines, for example those running multiple tracks at high speeds. The Print/Label application 514 can also be implemented to provide pre-printed labels in accordance with the illustrative embodiments herein. The Print/Label application 514 for pre-printed labels functions similarly in terms of receiving codes requests and delivering codes, however the codes are sent directly to local printers, instead of being sent remotely, for example, to a production facility or manufacturing plant. For security reasons, the labels can be shipped in an “active” state, and are only activated for verification once the plant representative (or other previously selected individual) confirms receipt of the labels. The labels can incorporate both overt and covert security features for enhanced protection against counterfeiting of medicines, as described in greater detail herein with reference to FIG. 11.

In accordance with an illustrative embodiment, the serialization platform can also provide a Reconcile application 515, in which the serialization platform 510 reconciles the printed unique codes with requests for codes and tracks the production statistics.

The serialization platform also provides for multi-level serialization 520, in which the serialization is performed for multiple levels (e.g. stages or checkpoints) in packaging and/or production of the pharmaceutical products. A multi-level serialization application 520 also allows interested users (such as an operations manager or supply chain manager) to “track-and-trace” production of the products. By serializing at multiple levels during production, it is possible for the production to be tracked and traced using the authentication codes from the serialization. The multi-level serialization can additionally allow for authentication codes on individual products themselves, in addition to authentication codes on packages (i.e. groups) of products. Multilevel serialization 520 further allows for tracking of production, to determine when the individual products have been allocated with a code, and also determine when a package of products has been allocated with a code. This allows the authentication codes to have the dual purpose of verifying the individual product while allowing managers and other persons to track and trace the production and/or printing of codes on pharmaceutical products. The track-and-trace can occur through an EPCIS (Electronic Product Code Information Service) or other repository that provides a unique, serialized identifier for an object. The track-and-track output can be in any one of a variety of standard formats known to those ordinarily skilled in the art. The serialization platform also provides Enterprise Resource Planning 522, an application that connects to existing Enterprise Resource Planning (ERP) implementation and retrieves production planning data that is used to generate the correct number of codes prior to run-time production. The generation of codes can thus become automatic, without (“free of”) manual intervention, and the associated overhead and potential for human error. The ERP module 522 is customizable to be compatible with a variety of existing ERP platforms, as should be apparent to those having ordinary skill

In accordance with the illustrative embodiment, the serialization platform 510 can further includes a Mobile monitoring module 524 which provides secure, real-time production status on a mobile device. The mobile monitoring module 524 empowers operations and supply chain managers to monitor the current status of packaging and printing across manufacturing lines and across different plant locations. This allows operations and supply chain managers, and any other interested individuals, to instantly detect any production issues and intervene as needed. The mobile monitoring application can also be employed by a plant supervisor or other similar individual to monitor production status without the need to be physically present at a production facility.

The serialization platform also provides an intuitive tool for real-time quality control, the Quality assurance module 526. The quality assurance application 526 immediately reads an authentication code and verifies that it has been printed correctly and is functioning as expected. This can be performed as a mandatory step prior to quality assurance approval of a production lot, and the results can be printed out and attached to an approval (e.g. “signoff”) report for record-keeping purposes.

In run-time operation of the system, requests for codes and other data are transmitted from the production facility platform 535 via datastream 537 to a network 530, and then via datastream 539 to the serialization platform 510. Codes and other pertinent data are then transmitted from the serialization platform 510 via datastream 528 to the network 530 and then via datastream 532 to the production facility platform 535. The production facility platform can be a platform, dedicated computer or server at the facility, or just a standalone printing application at a printer, within the facility or remote from the facility. The network can comprise any appropriate communication network including a local area network (LAN), the broad worldwide Internet, cellular telephones and smart phones used through a cellular tower network or any other appropriate network or secured connection known in the art.

During run-time operation of the system, the serialization platform 510 can be accessed by a production facility platform 535 or other entity interested in receiving codes for printing or otherwise affixing authentication codes to pharmaceutical products or other objects. The serialization platform 510 does not require all of the serialization tasks shown in FIG. 5, nor is it limited to the applications shown, as the applications or tools shown and described are for illustrative purposes as being representative of available features of the serialization platform. Through the serialization platform, it is possible to perform any one (or more) of the tasks associated with serialization of the products.

The production facility platform 535 also provides production data and printing status data (i.e. whether or not a code has been printed) via datastream 537. This information is used in verification of a product once requested by a user, and also used by several of the applications implemented by the verification platform, both to verify the authenticity of the code, as well as to provide intervention and/or visualization(s) as appropriate.

The code for a product (which is typically the product itself or a pre-printed label in some embodiments) is sent to a user, either via datastream 540 or physically through appropriate shipping systems known in the art. During run-time operation of the system, a user 542 submits a request for verification of a product via datastream 544 to the network 530 and then via datastream 546 to the verification platform 550. The verification platform 550 can be the same as the verification system 150 shown in FIG. 1, in addition to the system 150 shown in FIG. 1, or as a separate verification platform. The verification platform includes at least one of a plurality of modules 1557 for verifying the verification request received by the user. The verification platform can implement one or more of the verification modules 557 for verifying an authentication code, and can provide the user with the option of selecting the preferred method of verification for the user.

In accordance with an illustrative embodiment, the verification platform 550 includes a Text verification module 551, in which a text message (e.g., SMS (Short Message Service) message) is used for verification of medicines using codes. For example, an SMS message sent to a cellular telephone, smart phone or other mobile device. The text verification operates by a patient (user 542, for example) typing the code printed on the package and sending a verification request (via datastream 544) to a number identified on the product. The verification request can be either to a longcode, premium shortcode, or toll-free shortcode based on the manufacturer preference and regional telecom regulations. Within a few seconds, the user receives a customized response, also by text message, indicating whether the code is authenticated, expired, or oververified or invalid as per the settings selected by the production facility team.

The verification platform 550 can illustratively include a Voice module 552 through which users can speak with a person, rather than entering a code by text message or on the web (Internet or other network). The Voice module 552 is a multi-lingual, extended-hour verification service that uses highly trained agents to walk a patient through the verification process and help resolve any issues they may encounter. The agents of the Voice module verification service are further able to receive feedback from customers that assists in monitoring a pharmaceutical product, which can be particularly useful for certain populations of patients, such as the elderly and those in certain emerging markets.

A mobile application 553 can be implemented by the verification platform as a means by which an authentication code is verified. The mobile application 553 provides code verification of medicines for an Android platform or iOS (iPhone operating system) platform, or other mobile device computing platform, through use of a mobile application. Using the mobile application 553, users can type in a code and get a response within seconds. The user can also use the camera of their phone or mobile device (such as a tablet or laptop) to scan a barcode and have it verified almost instantly. A complete history of authentication requests is available on the application, enabling users to track their medicine purchase(s).

The verification platform 550 can further include a web application 554 for web-based (e.g. Internet) verification of medicines when authentication-enabled track-and-trace. The web application 554 has at least two user-specific types of applications: 1) directed at a user (patient) and 2) directed to supply chain intermediaries. The web application 554 allows patients (users) to quickly authenticate codes, and also allows supply chain intermediaries to scan tertiary, secondary or primary packages to both verify them and facilitate tracking of these products to their destinations. Authentication-enabled track-and-trace allows supply chain intermediaries to have more control of their supply chain, and is enabled once a medicine is authenticated either by a user or by being present as a particular package (tertiary, secondary or primary) or at a particular location (e.g. checkpoint) during production and/or distribution of the pharmaceutical product.

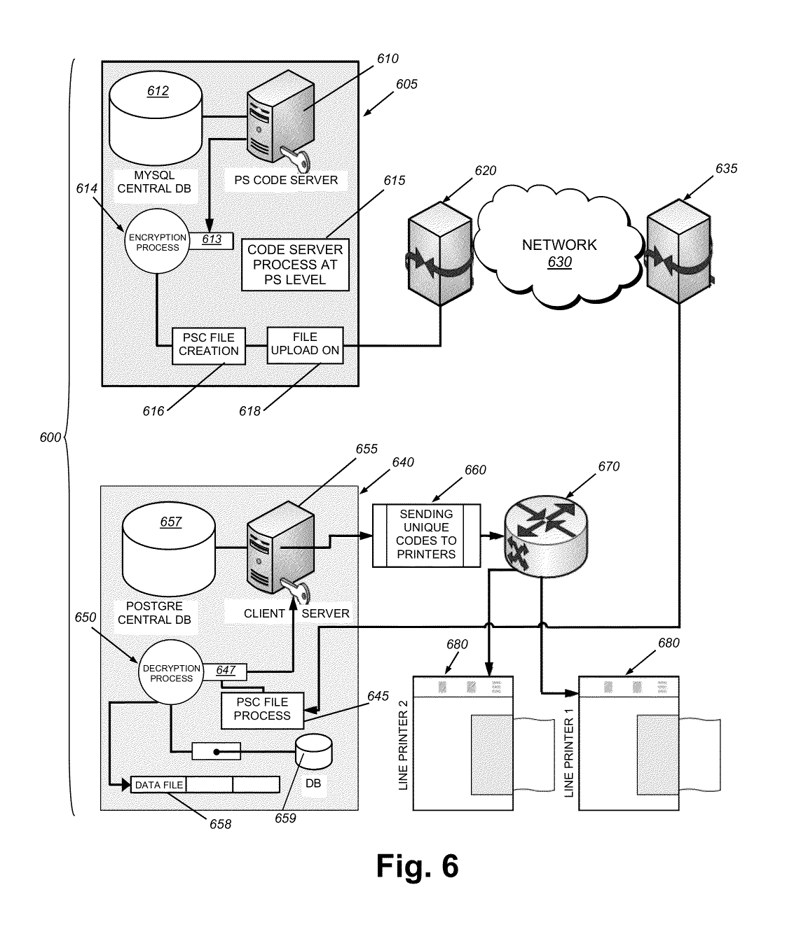

In an illustrative embodiment, the verification platform 550 can participate in IGR (INTERPOL Global Register) module 555, which is used for verification of the pharmaceutical products. The verification platform 550 has a secure link to the IGR system allowing customers to use the powerful IGR network. This verification can be in addition to, or instead of, the verification database shown and described herein (for example, verification database 155 in FIG. 1 and code database 612 in FIG. 6).

The verification platform 550 can further include an Alerts/Interventions application 559 for providing real-time alters and interventions when something unexpected occurs in the supply chain. The alerts/interventions application 559 can provide alerts when an unexpected error occurs and the assistance needed to address the problem immediately. The verification platform 550 employs a group of individuals to assist supply chain managers and other individuals to establish a proprietary set of rules and conditions to detect potential threats and trigger alerts or change user response “on-the-fly” (during runtime system operation). The verification platform 550 can be used in combination with the voice application 552 to direct a call-back to a patient to gain more information about the event in question and take appropriate steps as needed. Positive responses can also be triggered by the alerts/interventions application 559, such as loyalty bonuses for loyal patients (users), product-specific promotions or invitations to try other related products.

A visualization application 561 can also be employed by the verification platform 550 to perform bespoke (i.e. made-to-order or custom made) distribution and verification visualization. As patients verify the products, producers of pharmaceutical products can view particular details for a particular product, a particular lot, within a set time period, from a particular geography, or any combination thereof. Trend analysis can be used to better understand patient behavior across periods of time and indicate whether certain products are more in demand than others. The distribution and verification visualization can also be used to analyze time-to-market across geographies and products, indicate regional stock outs and afford better oversight of product distribution networks.

Reference is made to FIG. 6 showing a flow diagram for generating and printing code data within the overall printing system 600. In an illustrative embodiment, the data transfer occurs at multiple levels in the application covering the central server environment 605, the client server environment 640 and including the printing environment 670. Although the diagram depicts a separate code server and associated database and a client server and associated database, it should be readily apparent to those of ordinary skill that the various tasks and applications can be distributed among the system entities as appropriate to achieve the desired functions herein. Moreover, the central code server 610 can be a separate server and/or application or can be incorporated into an existing verification system (i.e. 150 of FIG. 1 or verification platform 550 of FIG. 5) or as an additional server that is part of the verification server 150 (or platform 550) or can be part of the management server (i.e. 160 of FIG. 1). The verification system can further comprise an IGR interfacing application for registration and verification through the IGR (INTERPOL Global Register) or an EPCIS (Electronic Product Code Information Service) or similar standard format for registration and verification. In further embodiments, all server tasks and applications can be combined into a single computing device or application, or can be spread over a series of servers and applications, as readily apparent within ordinary skill.

In an illustrative embodiment of the printing system 600, the codes are available at the central server 610 and are stored in a central database 612. According to the illustrative embodiment, the central codes database 612 is a PostGRE SQL server for the client application. Other database and server technologies within ordinary skill are expressly contemplated and can be employed. The central database files can be copied and hosted on any server on any platform, by copying and pasting on the prefixed location. Employing this option allows any user at the client end side 640 to open the database and see the codes that either have been printed or will be printed on the pharmaceutical products. Advantageously, a PostGRE SQL server at the client end allows this option to be employed by any user at the client end 640. This provides enhanced security because a user needs to be authenticated into the system, via password or other appropriate mechanism, for a particular database in order to access the contents thereof. Additionally, the PostGRE SQL server provides a stable and compact database, and users cannot open or host flies on any other server if they cannot authenticate to the database.

In accordance with an illustrative embodiment, the data flow of the codes are transferred from the code server 610 to the client level code server using a file transfer (connection 620 for the central server and connection 635 for the client server—or equivalent) over a network 630. The file 613 (for example a “.PSC”, secure code file, in an illustrative embodiment) is encrypted by an encryption process 614. The secure file is created 616 and uploaded at 618 to the network. A secure connection over the network 630 is established via the central server secure connection 620 and the client server secure connection 635. The encryption algorithm 614 encrypts the files so that a general user will not be able to see the file contents. This enhances security by ensuring that only certain entities are able to view the unencrypted file data. The code data is then transferred to the client server environment 640 via the client secured connection 635. The client has its own user name and password (or other authenticating information, such as a digital certificate) to access the network 630 or other secured connection to download the files. There is a file process 645 at the client environment 640 to import the file. The code 647 then undergoes a decryption process 650 so the codes can be stored by the client server 655 into the client database 657. The decryption process also decrypts the codes to generate and store a datafile 658 containing pertinent information regarding the code data.

Note, as used herein the term “process” can refer to any software and/or hardware process. In describing a particular process herein, the process can be embodied by a single functional block or a plurality of functional blocks, which can, in whole or in part, operate the particular process. Likewise, a given processor for performing a process can be embodied in one or more functional blocks, in whole or in part.

The client server environment 640 includes a printing database 659 that has been used for handling actual printing operation using printers at the production node. The client database 657 can also be used to obtain codes from the main server and perform uninterrupted operation even if the local area network 630 is unavailable. The printing database can also be used to send codes to remote locations for printing. The customer software as shown and described for the print/label application 514 with reference to FIG. 5 allows for remote printing. The client database 657 has sufficient codes for the production line and the codes are synchronized with the server by marking flags (or equivalent memory or software/hardware components) as “printed” or “not printed” during the print operation. Database technologies used, according to an illustrative embodiment, can include a PostGRE database technology which is a core backend database for the server at the client side environment 640 used as a direct connection to the server, for example as available from the PostgreSQL Global Development Group, Version 8.4.8. The database technologies can also include the MSAccess database technology available by Microsoft, version 2003 or any other readily applicable version, which is a database for obtaining codes operation at runtime, and is used through the Microsoft Jet engine in an illustrative embodiment.

According to an illustrative embodiment, the codes are sent from the client server 655 via step 660 to the printer application 670 that instructs the codes to be printed by the printers 680. The codes can alternatively be sent to a remote location or stored in a local or remote database for later use or printing. The user sends the codes as desired in any size connected to the server machine. The process then marks the codes as printed after receiving confirmation from the printer that the codes have been printed. The system 600 can also create the password protected database for printing to any variety of printers and printing applications. The user can define the number of codes desired to be exported to create the database. The user uses this database at the time of printing, so that the database is linked for variable field at the time of message creation to print any size of codes available in the code server database.

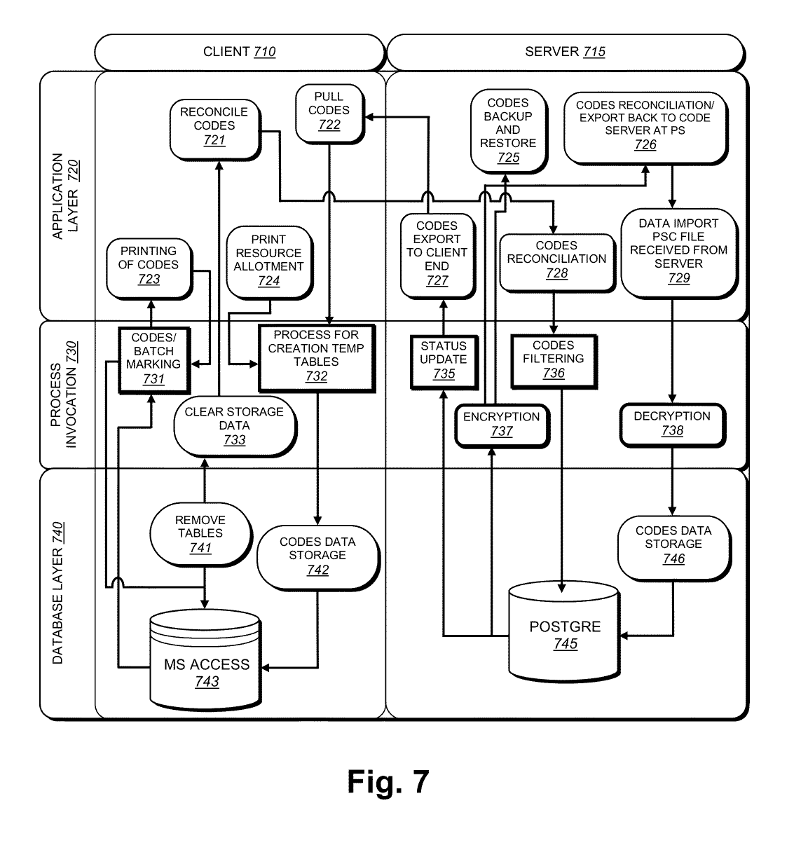

FIG. 7 illustrates a more detailed diagram of the client-server architecture of the printing application according to an illustrative embodiment. The architectural diagram of FIG. 7 shows the various operations performed by the client 710 and the server 715 at each of the application layer 730, the process invocation 730 and the database layer 740 for the overall system. As shown, the client 710, at the application layer 720, is responsible for reconciling codes 721, pulling or obtaining codes 722, printing codes 723 and printing resource allotment 724. The server 715, at the application layer 720, is responsible for codes backup and restore 725, codes reconciliation/export back to the code server 726, codes export to client end 727, codes reconciliation 728 and data import secure file receipt from server 729. The client 710, at the process invocation layer 730, is responsible for codes marking 731, the process for creation of temporary tables 732 and to clear storage data 733. The server 715, at the process invocation layer 730, is responsible for status update 735, codes filtering 736, encryption 737 and decryption 738. Encryption and decryption are only one example of the process(es) for safely and securely delivering codes. Any process for securely providing the codes can be employed and should be readily apparent to those having ordinary skill. More particularly, the server 715 is responsible for import, and where needed encryption, of data from the server database 745 and to the codes backup and restore 725 and codes reconciliation/export back to code server 726. Additionally, the server is responsible for export, and where needed decryption, of data import received from the server to codes data storage 746 into the server database 745. The client database 743 is accessed by the client 710 to produce codes marking 731 and also for codes data storage 742. The removal of tables 741 is also performed at the database layer 740. Codes exported from the server 715 to the client end (at 727) are transmitted to the client 710 so the client can reconcile the codes 721 with the codes reconciliation 728 at the server 715.

Collection of Realtime Market Data

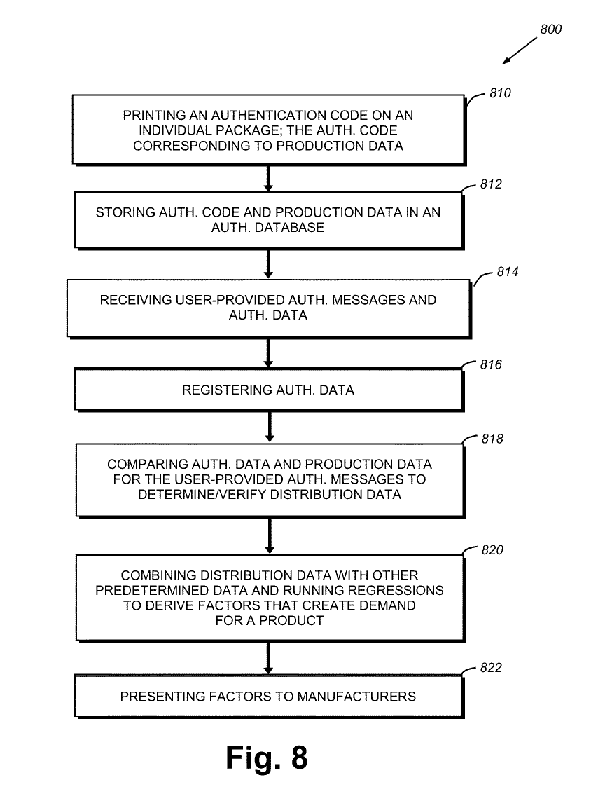

FIG. 8 shows a flow chart of a procedure for collecting and disseminating real-time market data of product distribution, according to the illustrative embodiments. As shown, the procedure 800 commences at step 810 by printing an authentication code on an individual package. The authentication code corresponds to production data pertaining to the production of the individual product and can also refer to the production and/or packaging of a group of products packaged into a larger package. Production data can include the time and place of production, drug information, and product batch information. At step 812, the authentication code and production data is then stored in an authentication database. At step 814, the procedure continues by receiving user-provided authentication messages and authentication data. As described hereinabove, the user can employ any one of a variety of different modalities for providing the message, including text message, SMS, MMS, Twitter tweets, USSD and other messaging services and standards. The authentication data can comprise the location, date, time, phone number, IP address, twitter handle, or other identifying information for the consumer. At step 816 the authentication data is then registered.

The procedure 800 continues to step 818 by comparing the user-provided authentication data and the corresponding production and/or identification data for the user-provided authentication messages to determine and/or verify the product or other identification. This allows a user to verify whether they have an accurate medicine or product, and also allows the distribution data to be used for further processing. At step 820, the distribution data can be combined with other predetermined data, such as disease incidents, and regressions are run on the combined data to derive factors that create demand for a product. These factors can then be presented to manufacturers at step 822. Information about the distribution and demand for a product is presented to the manufacturers either in real-time or in aggregated form for sale or licensing or on a dashboard. This allows manufacturers and other interested individuals to observe the factors that create a demand for a product.

Consumer Research and Communication

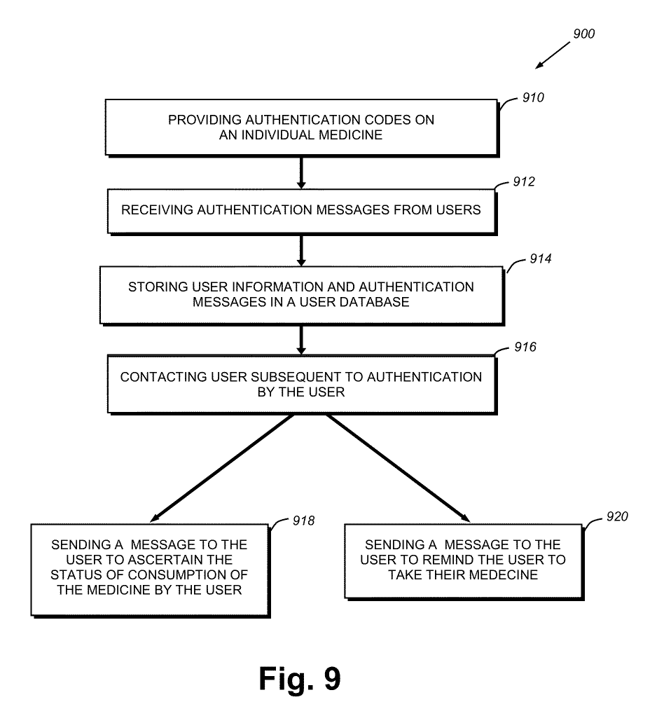

Reference is now made to FIG. 9 showing a flow diagram of a procedure 900 for conducting consumer research and communication, according to the illustrative embodiments. The procedure 900 commences at step 910 by providing authentication codes on an individual medicine. The individual medicine can be a particular medicine or product, such as a bottle, container, or other package as described herein and known in the art. Next, authentication messages are received from users at step 912 which provide information relating to an individual medicine which the user desires to authenticate. The user information and authentication messages are stored in a user database at step 914. The user information can include the IP address, phone number, Twitter handle and other information as desired. Next, at step 916, once a user has authenticated their medicine, the procedure contacts the user. This can be performed by either performing step 918 or step 920 which both send a message to the user. Step 918 sends a message to the user to ascertain the status of consumption of the medicine by the user. For example, a message can be sent by phone, SMS, email, or other appropriate message system, inquiring with the user as to whether they are still taking the medicine, have stopped taking the medicine, or have switched to a different brand name. As an example, a blood pressure patient purchases a medicine and authenticates it through authentication messages. Because the exact package, batch, drug, and date of authentication is known, a call or SMS message can be sent to the consumer a specified amount of time after the first authentication. This message can be used to determine if the consumer is still taking the medicine, if they have stopped taking it, or if they have switched to a different brand. This information is extremely valuable to manufacturers and brand owners and this is a novel way of interacting with consumers.

A message can also, or alternatively, be sent to the user at step 920 to remind the user to take their medicine. This is particularly useful in TB (tuberculosis), AIDS (acquired immune deficiency syndrome) and blood pressure medicines. This also allows manufacturers and other interested individuals to communicate directly with users anonymously, in that the manufacturers do not gather any personally identifying information, when so appropriately desired. A manufacturer is able to specify the types of messages that are sent to a user upon authentication, without having any personally identifying information pertaining to any users.

Providing Realtime Interaction with Consumers

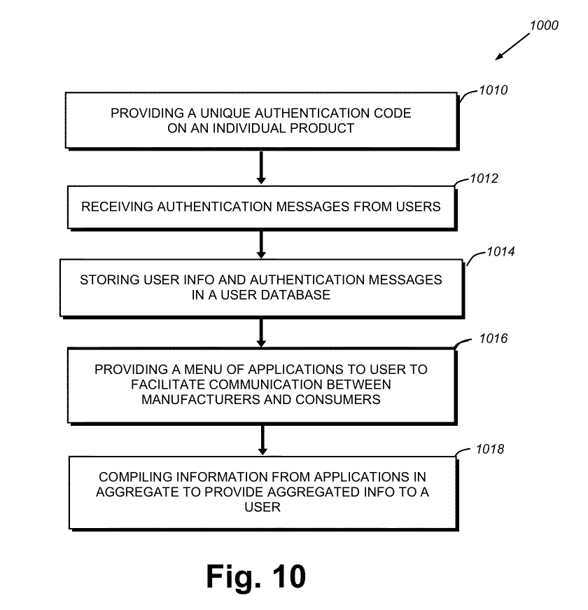

Reference is now made to FIG. 10 showing a flow diagram of a procedure for providing real-time interaction with consumers, according to the illustrative embodiments. The procedure 1000 commences at step 1010 by providing a unique authentication code on an individual product. The term product can refer to a medicine or other pharmaceutical product known in the art, and is described as a product for illustrative purposes. At step 1012, authentication messages are received from users, which allow a user to authenticate the particular medicine that a user has purchased. User information, including information corresponding to the user, and authentication messages are stored in a user database at step 1014. At step 1016 the procedure continues by providing a menu of applications to the user or the manufacturer to facilitate communication between manufacturers and consumers. The phone numbers and other identifying information is not shared with the manufacturers or other parties. A manufacturer chooses from the menu of applications, some of the applications being prepared by the system as described hereinabove, or can be licensed by third parties through which messages and interactions with the user can be initiated. The applications can be used to remind patients to take their medicines, to follow up with consumers about their product use, or provide consumers with more information about their drugs or the interactions between drugs. Any application can be provided in the menu of applications that facilitates communication between manufacturers and a sampling of consumers (users) immediately over a period of times. The information from the applications is then compiled in aggregate at step 1018 to provide aggregated information to the user and/or the manufacturer. The results from the applications can be compiled specifically, and including personal identifying information, or compiled anonymously, with no identifying information being provided. The aggregated information is provided to the manufacturer to further improve their interaction with users.

Covert Authentication

In accordance with the illustrative embodiments herein, the code data and/or label including the code data can incorporate a covert authentication element into the printing and/or providing of independent authentication codes to products. The covert authentication element can be constructed and arranged to be tested by the user, or to be sent to a remote location, or a message to a remote user containing instructions for where to find and/or test the covert authentication element. For example, the codes can be printed using a special type of ink or on a special paper that provides an additional layer of security in further embodiments. According to this covert embodiment, an additional layer of authentication is provided that is applied during the printing process and can also, independently, be verified. For example, the code data can be printed using a special ink or other chemical incorporated therein that can be tested by the user or sent to a remote location for testing, to verify the authenticity of the pharmaceutical product. The cover element employs a particular structure or chemical, such as a chemical tag in the ink, which can be tested to determine whether the substance or chemical is present in the ink. This provides a dual-authentication system when combined with any other overt authentication or identifying code procedures as described herein.

FIG. 11 is a flow diagram showing a procedure for performing dual-authentication using an authentication code and a covert authentication element, according to the illustrative embodiments. As should be readily apparent to those having ordinary skill in the art, the steps of procedure 1100 shown in FIG. 11 can be performed in any order after the authentication code is printed which includes a covert element on an individual product. At step 1110, the procedure commences by printing a unique authentication code including a covert authentication element on an individual product. Thereafter, the procedure receives an authentication message containing authentication data from a user at step 1112 and then verifies the authentication data at step 1114, and verifies the covert element at step 1116. Steps 1112 and 1116 can occur at the same time, or at different times. The procedure verifies the authentication data at step 1114 and proceeds to step 1118 if the authentication data is not verified, and a user can be sent a message that the medicine was not verified. Verified authentication data proceeds to step 1122 at which a verification and response is sent to the user. Similarly, the verify covert element step 1116 results in a not verified result 1120 or a verification and response message at step 1122. At step 1120 a message can be sent to the user that the product has not been verified. At step 1122 a message authenticating the product is sent to the user.

As an additional or alternative security measure, labels can be shipped in an “inactive” state such that they are not “active” until the labels reach their desired location and are activated. The activation can be done manually by a manager or other individual at the printing location, or can be done automatically once the label arrives at a particular location and is verified to be at that location. The inactive labels are another covert authentication at step 1116 to verify the covert element. The label needs to be “active” (e.g., by having been activated by a manager or other individual at the printing or production facility) in order to be properly verified. Thus, if the code numbers are somehow compromised, they will still not be valid if they are not activated. The security of the overall system and efforts against counterfeiting are enhanced by providing an inactive label that is activated upon printing, or in a close proximity to the printing of the labels.

Providing Information Upon Authentication

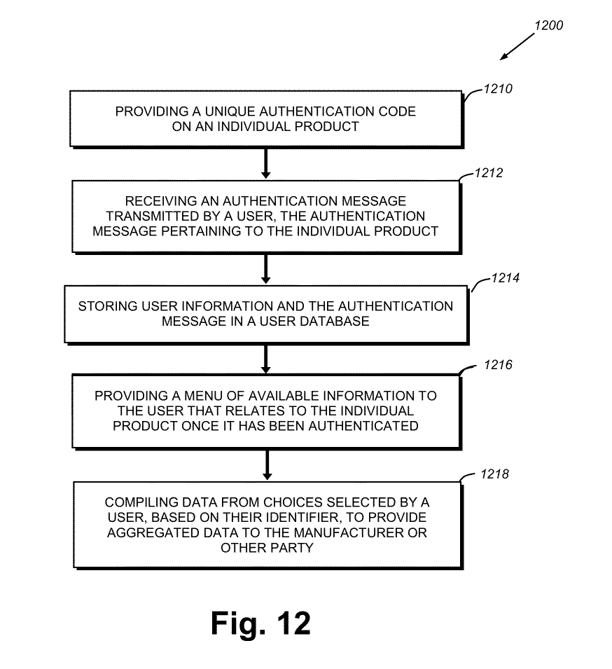

Reference is now made to FIG. 12 showing a flow diagram of a procedure for providing real-time information to users upon authentication of a product, according to the illustrative embodiments. The procedure 1200 commences at step 1210 by providing a unique authentication code on an individual product, in accordance with the illustrative embodiments described herein. The procedure then continues by receiving an authentication message that is transmitted by a user at step 1212. The authentication message pertains to the individual product that a user desires to authenticate. User information and the authentication message are then stored in a user database at step 1214. Once the individual product has been authenticated, at step 1216 a menu of available information is provided to the user. The menu of available information relates to the individual product. For example, if the medicine is to treat hypertension, information would be provided to the user as to what hypertension is, ways to prevent hypertension, support available, drugs that interact with this drug, and other pertinent information. This is highly variable and customizable depending upon the particular product that is authenticated by the system. Then at step 1218 data from particular choices of information selected by a user are compiled to provide aggregated data to the manufacturer or other party. This allows manufacturers and other parties to become aware of the selections of a user based upon the medicine or other product that they have identified. Furthermore, the data is compiled based upon the user identifier, which can be an anonymous identifier, to protect the privacy of the user, or can be the telephone number, IP address, Twitter handle, or other appropriate identifier.

Exemplary Screen Displays

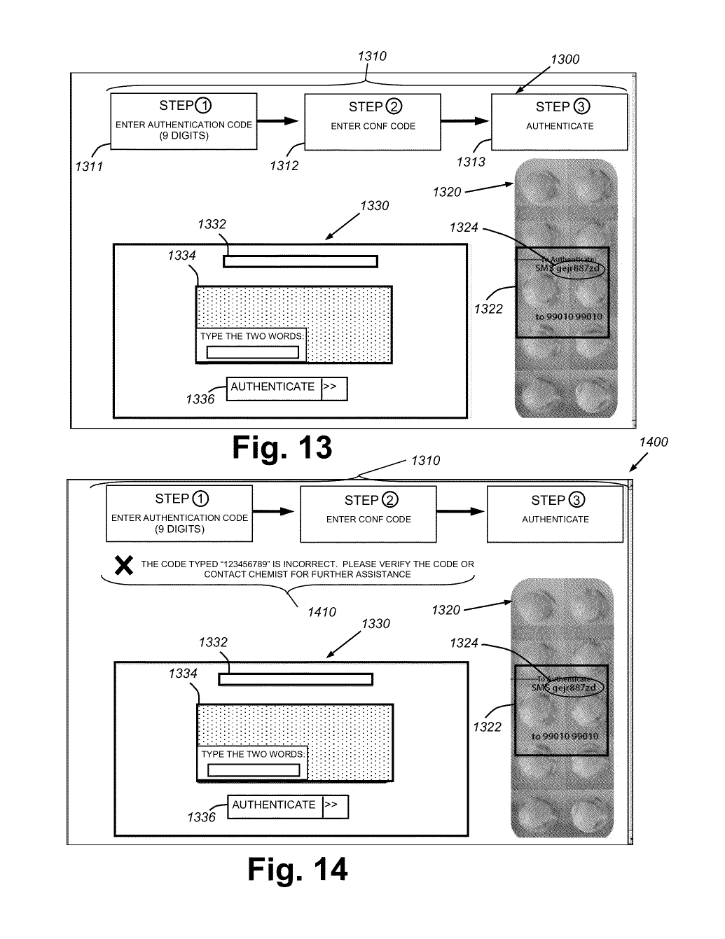

Reference is now made to FIGS. 13-16 showing exemplary screen shot displays for authentication of a particular product, for example as a display on a phone through SMS messages, or as a web portal display, or other appropriate display for sending and receiving information relating to a particular product. FIG. 13 shows an exemplary screen display 1300 which shows the procedure 1310 for authenticating a particular medicine. As described hereinabove, the procedure commences at step 1311 where the user enters an authentication code, then at step 1312 enters a confirmation code, and at step 1313 the product is authenticated, or another appropriate message is transmitted to the user. As shown in the display of FIG. 13, an exemplary product 1320 has instructions 1322 printed thereon with an authentication code 1324 (“gejr87zd”) which can be used to authenticate the product. A user viewing the screen display 1300 can enter various information into the entry box 1330, which includes the authentication code into authentication box 1332, a confirmation code entered into confirmation box 1334 and then a user selects the authenticate box 1336 to determine the authenticity of their product. Note that the step of entering a confirmation code is optional and does not need to be performed in all embodiments to perform authentication of a particular product. The confirmation code can be an externally provided service, such as the publically available reCAPTCHA and corresponding CAPTCHA codes, or can be another appropriate system for verifying that an actual user is entering the authentication codes. If an appropriate and verifiable code is provided, a user is provided with one of a series of screen displays, although not shown, which provide a user with applications, information pertaining to their product, or other information and messages in accordance with the illustrative embodiments described herein.

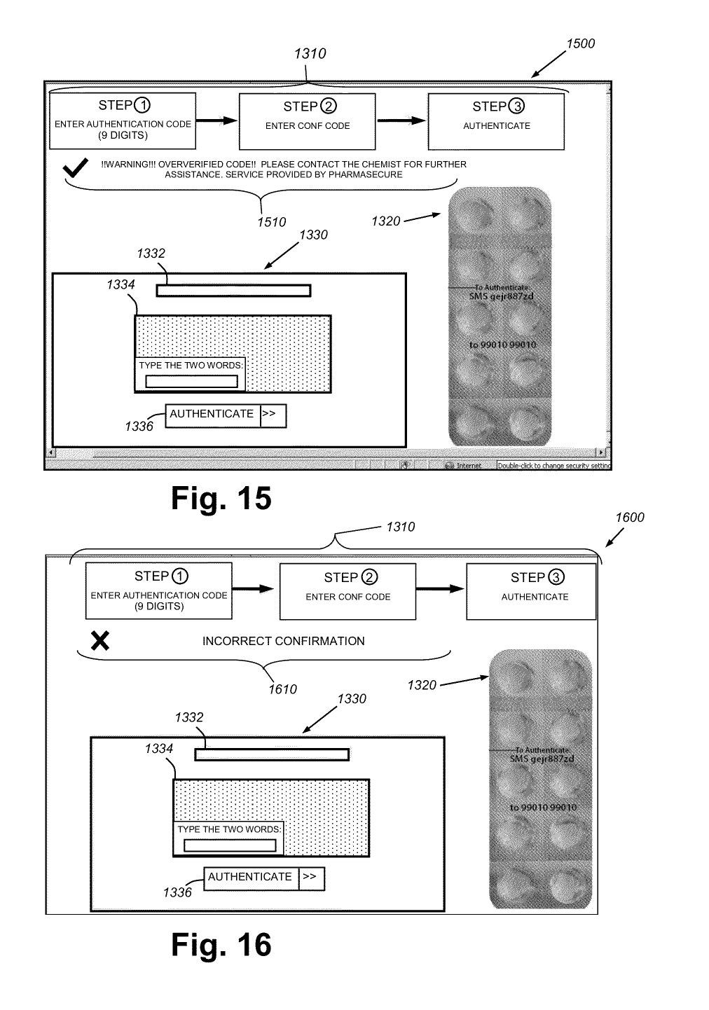

If an incorrect code is typed, a user is directed to an exemplary screen display such as the display 1400 in FIG. 14. As shown, an error code 1410 appears to notify the user that the code they have typed is incorrect. The user is further instructed to either verify the code or contact a chemist or local pharmacist for further assistance. If an oververified code is typed, meaning a code that has already previously been verified, the user is directed to an exemplary screen display such as display 1500 of FIG. 15. As shown, a message 1510 is presented to the user, thereby warning the user that the code ahs been oververified. This indicates to a user that the product could be counterfeit or damaging in some other way. The user is further warned by the user that a chemist or pharmacist should be contacted for further assistance. If a confirmation code is used, and the user enters an incorrect confirmation code, the user is directed to an exemplary screen display such as the display 1600 shown in FIG. 16. The user is presented with an error code 1610, as shown, which indicates that an incorrect confirmation has been entered. Thus the user is invited to re-enter the data in the data entry box 1330, including entry fields 1332, 1334 and 1336, to attempt to authenticate the product. These exemplary screen shot displays are for illustrative purposes only and are highly variable within ordinary skill to achieve the functionalities described herein.

The systems and methods described hereinabove have been described primarily with reference to an individual bottle of medicine and a unique authentication code provided thereon for user authentication. However, it is expressly contemplated that authentication codes can be provided and printed on any package of medicine, including an individual medicine and a package containing a plurality of medicines, and also authentication codes for an overall shipment of medicines. These authentication codes can be authenticated by a plurality of users in a system, according to the teachings herein, to verify production data and other information pertaining to the pharmaceutical products.

The teachings herein and other applications and advantages should be clear to an ordinarily skilled person. The systems and methods herein improve review of sales and medicines and the communication between a pharmaceutical company and users of the medicines. Authentication of pharmaceutical products is also enhanced by this system by allowing users to ensure that they are purchasing and ingesting proper validated medicine and not counterfeit products.

The foregoing has been a detailed description of illustrative embodiments of the invention. Various modifications and additions can be made without departing from the spirit and scope of this invention. Each of the various embodiments described above may be combined with other described embodiments in order to provide multiple combinations of features. Furthermore, while the foregoing describes a number of separate embodiments of the apparatus and method of the present invention, what has been described herein is merely illustrative of the application of the principles of the present invention. For example, the procedures have been described with reference to managing medicines or particular drugs or other products provided by a pharmaceutical company. However, the teachings herein are readily applicable to any medicine that is sold or distributed, for which data is available. Additionally, the systems and applications herein are described as residing on a particular computing environment, database structure or client-server architecture. However, the type and arrangement or systems and components is highly variable within ordinary skill to achieve the functions described herein. In addition, any of the procedures, functions and/or processes described herein can be performed using electronic/computer hardware, software consisting of a non-transitory computer-readable medium of program instructions, or a combination of hardware and software. Accordingly, this description is meant to be taken only by way of example, and not to otherwise limit the scope of this invention.

What is claimed is:

1. A system for managing distribution of pharmaceutical products or medicines, the system comprising:
a central server operatively connected to a database that a plurality of authentication codes;
means for encrypting at least one of the plurality of authentication codes to create an encrypted code file;
means for decrypting the encrypted code file to create a decrypted code file; and
a printing application that receives the decrypted code file and instructs at least one printer to print the decrypted code file and transmit printing information to confirm that the decrypted code file has been printed.

2. The system of claim 1 wherein the means for encrypting comprises an encryption process running on the central server.

3. The system of claim 1 wherein the means for decrypting comprises a decryption process running on a client application.

4. The system of claim 1 further comprising means for storing an authentication code that has been printed on a product and the printing information in the database.

5. The system of claim 4 further comprising means for receiving at least one user-provided authentication message and associated authentication data from a user.

6. The system of claim 5 further comprising means for comparing the authentication data and the printing information for the at least one user-provided authentication message to determine distribution data.

7. The system of claim 6 further comprising means for combining distribution data with other predetermined data and running regressions to derive factors that create demand for a product so that the factors that create demand for the product can be presented to manufacturers of the product.

8. The system of claim 6 further comprising means for verifying the authentication data using Interpol Global Register.

9. The system of claim 5 further comprising:
means for storing user information and the user-provided authentication message in a user database;
means for providing a menu of available information to the user when the product has been authenticated, the available information related to the product; and
means for compiling data from choices selected by the user, based on an identifier of the user, to provide aggregated data to the manufacturer or other party.

10. A system for performing dual-authentication of medicines or products, the system comprising:
means for providing a unique authentication code for a pharmaceutical product, the unique authentication code including a covert authentication element;
means for receiving an authentication message containing authentication data from a user; and
means for providing a verification response to the user upon
(a) verifying the authentication data received by the user and
(b) verifying the covert authentication element.

11. The system of claim 10 wherein the covert authentication element comprises a label that is in an inactive state until activated by a production means.

12. The system of claim 10 wherein the covert authentication element comprises a testable ink or a testable paper.

13. The system of claim 10 wherein the means for providing a verification response comprises verifying the authentication data through Interpol Global Register.

14. A system for managing distribution of pharmaceutical products or medicines, the system comprising:
means for serializing a plurality of authentication codes used in verifying authenticity of pharmaceutical products, in response to a request for the plurality of authentication codes from a production means;
means for verifying at least one authentication code of the plurality of authentication codes, in response to a verification request submitted by a user of a pharmaceutical product.

15. The system of claim 14 wherein the means for verifying the at least one authentication code comprises using Interpol Global Register.

16. The system of claim 14 wherein the means for verifying the at least one authentication code comprises comparing the at least one authentication code to each of the plurality of authentication codes.

17. The system of claim 14 wherein the production means comprises a production server at a production facility and further comprises means for serializing the plurality of authentication codes at multiple stages in production of respective products at the production facility.

18. The system of claim 17 further comprising means for alerting a production manager at the production facility when the plurality of production codes are not at a correct stage of production.
